(19) United States
(12) Patent Application Publication
(10) Pub. No.: US 2024/0008944 A1
(43) Pub. Date: Jan. 11, 2024

(54) TREATMENT INSTRUMENT

(71) Applicant: OLYMPUS CORPORATION, Tokyo (JP)

(72) Inventors: Hisatsugu TAJIMA, Tokyo (JP); Noriaki Yamanaka, Tokyo (JP)

(21) Appl. No.: 18/471,965

(22) Filed: Sep. 21, 2023

Related U.S. Application Data
(63) Continuation of application No. PCT/JP2021/020608, filed on May 31, 2021.

(51) Int. Cl. A61B 34/00; A61B 17/29

(52) U.S. Cl. CPC A61B 34/71; A61B 17/29; A61B 2034/742

(57) ABSTRACT

A treatment instrument includes: a shaft; an end effector disposed on a distal side of the shaft; a joint connecting the end effector to a distal end of the shaft; a first part connected to a proximal side of the shaft and configured to bend the joint; wires each connecting the joint and the first part through the shaft and each configured to generate tension in response to an operation of the first part; and a second part connected to the proximal side of the shaft and configured to integrally rotate the shaft, the end effector, the joint, the first part, and the wires about a rotation axis passing through central axes of the shaft, the end effector, the joint, and the first part. The first part includes: a joystick displaceable in a direction intersecting a central axis of the shaft; and a lock fixing a position of the joystick.

### CROSS-REFERENCE TO RELATED APPLICATIONS

This is a continuation of International Application PCT/JP2021/020608 which is hereby incorporated by reference herein in its entirety.

### TECHNICAL FIELD

The present invention relates to a treatment instrument.

### BACKGROUND ART

A known treatment instrument for endoscopic surgery, represented by laparoscopic surgery, in the related art has a joint on the proximal side of a treatment part (for example, see PTL 1). The treatment instrument having the joint is suitable for treatment in a confined space because the posture of the treatment part can be changed only by bending the joint.

The treatment instrument described in PTL 1 includes a rotation operation part that rotates the treatment part about the central axis of the joint. The rotation operation part has a dial that is rotationally operated by an operator and a plurality of links arranged in series in the joint. A rotational force of the dial is transmitted to the treatment part by the frictional force between the plurality of links to rotate the treatment part. With this configuration, the treatment part rotates independently of bending of the joint. That is, it is possible to rotate the treatment part while maintaining the bending direction and the bending angle of the joint. Because such a treatment instrument can realize roll motion of the treatment part without providing a roll joint on the distal end, it is advantageous in terms of shortening the length of the treatment instrument and reducing the cost.

### CITATION LIST

### Patent Literature

{PTL 1} Japanese Unexamined Patent Application, Publication No. 2004-154164

### SUMMARY OF INVENTION

According to an aspect of the present invention, there is provided a treatment instrument including: a shaft; an end effector disposed on a distal side of the shaft; a joint connecting the end effector to a distal end of the shaft and being bendable in a direction intersecting a central axis of the shaft; a first operation part connected to a proximal side of the shaft and configured to bend the joint; a plurality of wires each connecting the joint and the first operation part through the shaft and each configured to generate tension in response to an operation of the first operation part; and a second operation part connected to the proximal side of the shaft and configured to integrally rotate the shaft, the end effector, the joint, the first operation part, and the plurality of wires about a rotation axis, which passes through the central axis of the shaft, a central axis of the end effector, a central axis of the joint, and a central axis of the first operation part. The first operation part includes: a joystick displaceable in a direction intersecting the central axis of the shaft; and a lock configured to fix a position of the joystick.

According to another aspect of the present invention, there is provided a treatment instrument including: a shaft; an end effector disposed on a distal side of the shaft; a joint connecting the end effector to a distal end of the shaft and being bendable in a direction intersecting a central axis of the shaft; a first operation part connected to a proximal side of the shaft and configured to bend the joint; a plurality of wires each connecting the joint and the first operation part through the shaft and each configured to generate tension in response to an operation of the first operation part; and a second operation part connected to the proximal side of the shaft and configured to integrally rotate the shaft, the end effector, the joint, the first operation part, and the plurality of wires about a rotation axis, which passes through the central axis of the shaft, a central axis of the end effector, a central axis of the joint, and a central axis of the first operation part. The first operation part includes a joystick displaceable in a direction intersecting the central axis of the shaft and movable on a plane extending in a direction perpendicular to the central axis of the shaft so as to be parallel to the plane.

According to further another aspect of the present invention, there is provided a treatment instrument including: a shaft; an end effector disposed on a distal side of the shaft; a joint connecting the end effector to a distal end of the shaft and being bendable in a direction intersecting a central axis of the shaft; a first operation part connected to a proximal side of the shaft and configured to bend the joint; a first wire, a second wire, a third wire, and a fourth wire each connecting the joint and the first operation part through the shaft and each configured to generate tension in response to an operation of the first operation part; and a second operation part connected to the proximal side of the shaft and configured to rotate the end effector about a central axis of the end effector. The first wire, the second wire, the third wire, and the fourth wire are held at four equally or substantially equally spaced positions about a central axis of the first operation part at the first operation part and are held at four equally or substantially equally spaced positions about a central axis of the joint at the joint. The first wire, the second wire, the third wire, and the fourth wire are gathered together in one place away from the central axis of the shaft in a direction intersecting the central axis, on the proximal side of the shaft.

### BRIEF DESCRIPTION OF DRAWINGS

FIG. 1A illustrates an overall configuration of a treatment instrument according to an embodiment of the present invention.

FIG. 1B illustrates operations of a bending operation part and a joint of the treatment instrument in FIG. 1A.

FIG. 2 is a perspective view showing a configuration example of the joint.

FIG. 3 is a vertical cross-sectional view of a connection member, a rotation operation part, and the bending operation part.

FIG. 4A illustrates the positions of four wires at the joint and the bending operation part.

FIG. 4B illustrates the positions of the four wires at the joint and the bending operation part after rotation of the rotation operation part.

FIG. 5 illustrates how to hold a grip with one hand.

FIG. 6A illustrates roll motion of an end effector.

FIG. 6B illustrates roll motion of the end effector.

FIG. 7A illustrates the positions of bending wires gathered together in one place at the connection member.

FIG. 7B is a front view of a second member viewed from a distal side.

FIG. 8A is a vertical cross-sectional view showing a modification of a shaft member of the bending operation part.

FIG. 8B illustrates a pressing force applied from a finger of an operator to a pressing part of the bending operation part in FIG. 3.

FIG. 9A is a side view of a modification of the bending operation part.

FIG. 9B illustrates a configuration of the bending operation part in FIG. 9A viewed from the proximal side.

FIG. 10A illustrates a configuration example of a lock mechanism.

FIG. 10B illustrates another configuration example of the lock mechanism.

FIG. 11A illustrates another configuration example of the lock mechanism.

FIG. 11B illustrates an operation of the lock mechanism in FIG. 11A.

FIG. 11C illustrates the lock mechanism in FIG. 11B viewed from another direction.

### DESCRIPTION OF EMBODIMENTS

A treatment instrument according to an embodiment of the present invention will be described below with reference to the drawings.

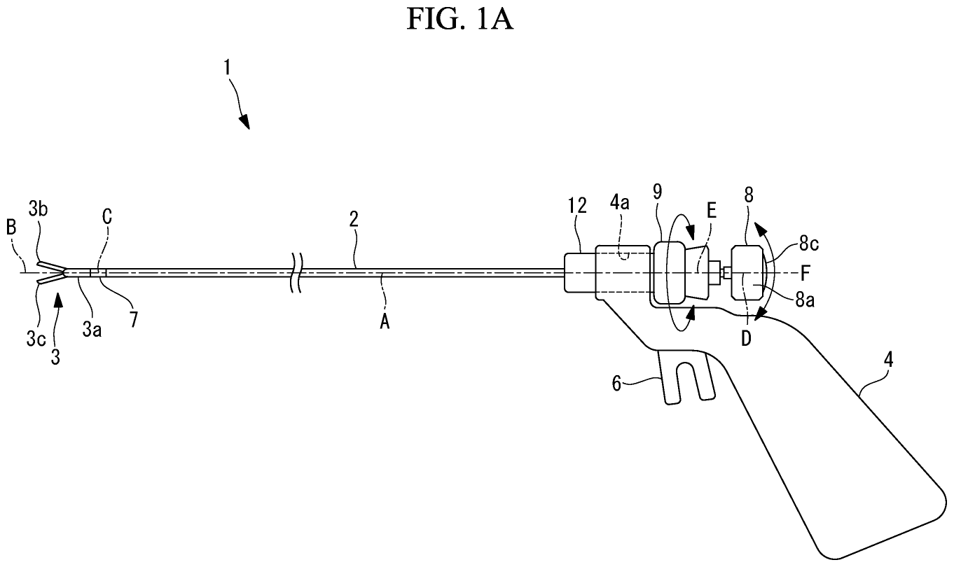

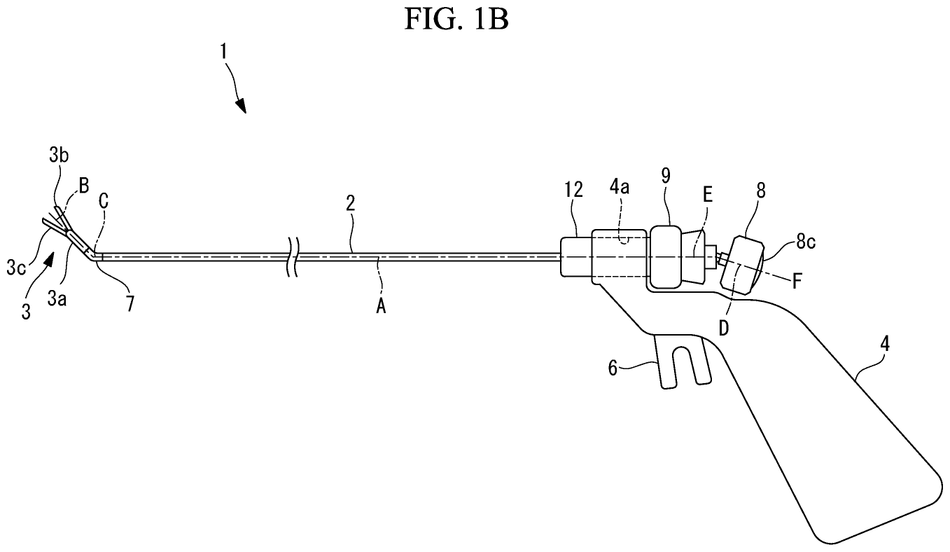

As shown in FIGS. 1A and 1B, a treatment instrument 1 according to this embodiment includes a long, rigid shaft 2, an end effector 3 disposed on the distal side of the shaft 2, and a grip 4 connected to the proximal side of the shaft 2 and to be gripped by an operator. The treatment instrument 1 has a vertical direction and a horizontal direction that are orthogonal to a longitudinal axis (central axis) A of the shaft 2 and orthogonal to each other. In FIGS. 1A and 1B, the direction perpendicular to the plane of the drawing is the horizontal direction.

The shaft 2 is a straight tubular member made of a rigid material, such as metal. A connection member 12 is connected to the proximal end of the shaft 2.

The grip 4 is shaped and sized to be suitable for an operator to grip with one hand. The connection member 12 is inserted through a hole 4a provided in the upper end of the grip 4, whereby the shaft 2 is supported by the grip 4.

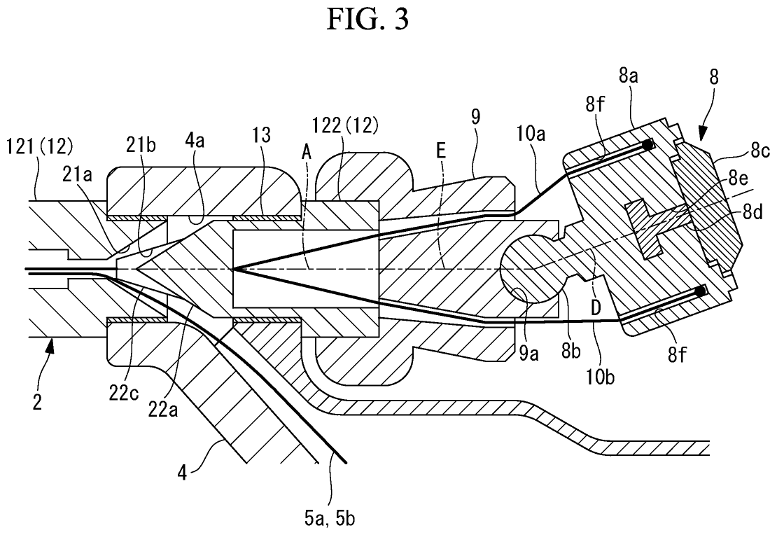

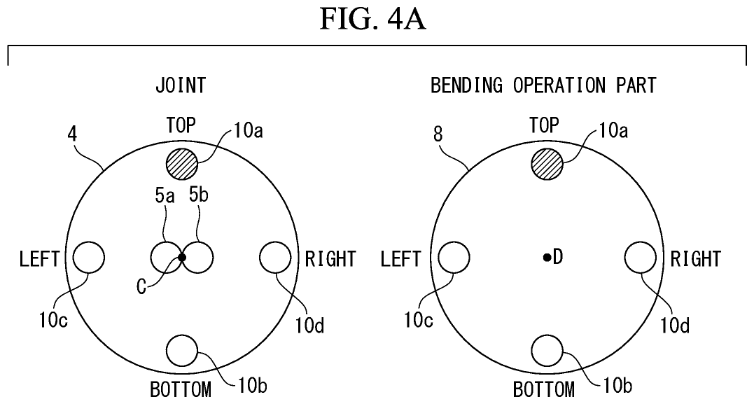

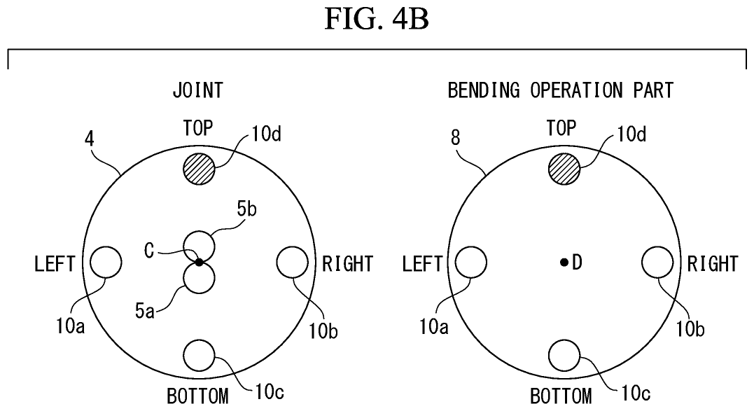

The end effector 3 is a treatment part that acts on tissue in a living body. In this embodiment, the end effector 3 is double-action forceps and includes a rigid frame 3a and a pair of jaws 3b and 3c supported by the frame 3a in a manner capable of opening/closing with respect to each other. Thus, the treatment instrument 1 further includes a pair of opening/closing wires 5a and 5b (see FIGS. 3 to 4B) for opening/closing the end effector 3, and a trigger 6. The trigger 6 is provided on the grip 4 and is connected to the end effector 3 by the opening/closing wires 5a and 5b passing through the inside of the shaft 2. The jaws 3b and 3c are configured to be opened or closed when the operator pulls the trigger 6, tensioning the opening/closing wires 5a and 5b.

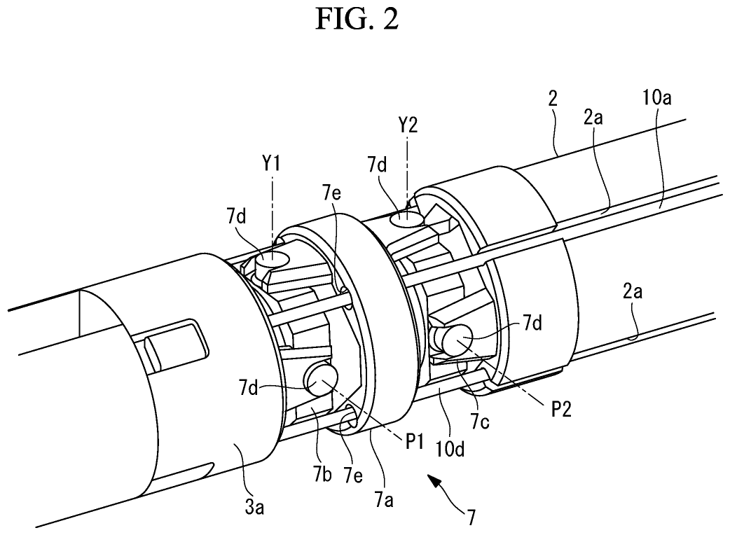

The treatment instrument 1 further includes a joint 7 coupling the end effector 3 to the distal end of the shaft 2, two operation parts 8 and 9 connected to the proximal side of the shaft 2, and four bending wires 10a, 10b, 10c, and 10d (see FIGS. 2 to 4B) connecting the joint 7 and the first operation part 8. The first operation part 8 is a bending operation part for bending the joint 7, and the second operation part 9 is a rotation operation part for rotating the end effector 3 about a central axis B of the end effector 3.

The joint 7 is disposed between the distal end of the shaft 2 and the proximal end of the end effector 3 and is bendable in any direction intersecting a longitudinal axis A of the shaft 2. For example, the joint 7 includes at least one pitch joint that can swivel about a pitch axis extending in the vertical direction and at least one yaw joint that can swing about a yaw axis extending in the horizontal direction. Alternatively, the joint 7 may include a ball joint.

FIG. 2 illustrates a configuration example of the joint 7 having the pitch joint and the yaw joint. The joint 7 in FIG. 2 includes a rigid ring-shaped joint link 7a and two ring-shaped intermediate members 7b and 7c disposed on the distal side and the proximal side of the joint link 7a, respectively. The joint link 7a is coupled to the intermediate members 7b and 7c by pins 7d so as to be swivelable about yaw axes Y1 and Y2. The intermediate member 7b is coupled to the proximal end of the frame 3a of the end effector 3 with the pins 7d so as to be swingable about a pitch axis P1, and the intermediate member 7c is coupled to the distal end of the shaft 2 with the pins 7d so as to be swingable about a pitch axis P2.

The bending operation part 8 is a joystick that can be displaced at least to the top, bottom, left, and right with respect to the longitudinal axis A of the shaft 2 and is connected to the proximal end of the shaft 2 via a rotation operation part 9. As shown in FIG. 3, the bending operation part 8 includes a base part (rotation part) 8a, a ball part 8b coupling the base part 8a to the rotation operation part 9, a pressing part 8c supported by the base part 8a, and a rod-like shaft member 8d with which the pressing part 8c is attached to the base part 8a. FIG. 3 is a vertical cross-sectional view taken along the longitudinal axis A of the shaft 2 and a central axis E of the rotation operation part 9.

The base part 8a is a columnar member.

The ball part 8b is provided on the distal side of the base part 8a and is rotatably supported by a spherical recess 9a formed in the proximal end face of the rotation operation part 9. That is, the ball part 8b and the recess 9a constitute a ball joint that couples the bending operation part 8 to the rotation operation part 9 such that the bending operation part 8 can swivel in any direction.

The pressing part 8c is a substantially disc-shaped member disposed at the proximal end face of the base part 8a so as to be coaxial with a central axis D of the base part 8a. An operator can tilt the bending operation part 8 in any desired direction by placing a finger (for example, a thumb) of one hand holding the grip 4 on the pressing part 8c and moving the finger.

The pressing part 8c is supported by the base part 8a by means of the shaft member 8d so as to be rotatable about the central axis D relative to the base part 8a. More specifically, the base part 8a has a hole 8e extending from the proximal end face of the base part 8a along the central axis D. The shaft member 8d inserted into the hole 8e is disposed coaxially with the base part 8a and is rotatable about the central axis D in the hole 8e. The pressing part 8c is fixed to the proximal end of the shaft member 8d. The pressing part 8c is lightly in contact with the base part 8a, or a clearance is provided between the pressing part 8c and the base part 8a.

The outer surface of the shaft member 8d is in contact with the inner surface of the hole 8e. Thus, friction occurs between the outer surface of the shaft member 8d and the inner surface of the hole 8e. Hence, when the base part 8a rotates about the central axis D in a state in which no pressing force is applied to the pressing part 8c, the shaft member 8d and the pressing part 8c also rotate integrally with the base part 8a due to the friction between the outer surface of the shaft member 8d and the inner surface of the hole 8e.

On the other hand, when a pressing force in a direction toward the ball part 8b is applied to the pressing part 8c, and the pressing force is stronger than the frictional force between the base part 8a and the shaft member 8d, the pressing part 8c and the shaft member 8d do not rotate, regardless of the rotation of the base part 8a. Specifically, the base part 8a rotates relative to the pressing part 8c, which is pressed and stopped by the finger, while resisting the friction.

Note that the configuration of the pressing part 8c is not limited to the above-described configuration. The pressing part 8c may be supported by the base part 8a by any method, as long as the base part 8a and the pressing part 8c are relatively rotatable.

The four bending wires 10a, 10b, 10c, and 10d extend from the joint 7 to the bending operation part 8 through the shaft 2, the connection member 12, and the rotation operation part 9 so as to be parallel to one another.

As shown in the left drawing in FIG. 4A, at the joint 7, the four bending wires 10a, 10b, 10c, and 10d are arranged at equal or substantially equal intervals in the circumferential direction about a central axis C, and the distal ends of the bending wires 10a, 10b, 10c, and 10d are fixed to the proximal end of the frame 3a. The bending wires 10a, 10b, 10c, and 10d pass through grooves 2a in the shaft 2 and holes 7e in the joint link 7a (see FIG. 2), thereby being held at four positions that are spaced apart from the central axis C in directions intersecting the central axis C and that are separated from one another by an equal or substantially equal distance about the central axis C. The bending wires (a first wire and a second wire) 10a and 10b are held on both sides of the central axis C, and the bending wires (a third wire and a fourth wire) 10c and 10d are held on both sides of the central axis C.

As shown in the right drawing in FIG. 4A, at the bending operation part 8, the four bending wires 10a, 10b, 10c, and are arranged at equal or substantially equal intervals in the circumferential direction about the central axis D of the base part 8a, and the proximal ends of the respective bending wires 10a, 10b, 10c, and 10d are fixed to the base part 8a. The bending wires 10a, 10b, 10c, and 10d are inserted into holes 8f (see FIG. 3) in the base part 8a, thereby being held at four positions that are spaced apart from the central axis D in directions intersecting the central axis D and that are separated from one another by an equal or substantially equal distance about the central axis D. The bending wires 10a and are held on both sides of the central axis D, and the bending wires 10c and 10d are held on both sides of the central axis D.

As shown in FIG. 1B, when the bending operation part 8 tilts in any direction, the bending wires 10a, 10b, 10c, and generate tension depending on the tilting direction of the bending operation part 8, causing the joint 7 to bend in the direction opposite to the tilting direction of the bending operation part 8.

The rotation operation part 9 is a dial-type handle disposed on the proximal side of the hole 4a in the grip 4 and is fixed to the proximal end of the connection member 12 passing through the hole 4a. The rotation operation part 9 is disposed coaxially with the longitudinal axis A of the shaft 2 and is rotatable about a central axis E of the rotation operation part 9 relative to the grip 4.

By rotating the rotation operation part 9 about the central axis E, the shaft 2, the end effector 3, the joint 7, the bending operation part 8, and the four bending wires 10a, 10c, and 10d integrally rotate about a rotation axis F.

The rotation axis F is an axis extending from the distal end of the end effector 3 to the proximal end of the bending operation part 8 and passing through the central axes B, C, A, and D of the end effector 3, the joint 7, the shaft 2, and the bending operation part 8. As shown in FIG. 1A, when the joint 7 and the bending operation part 8 are positioned at neutral positions, the rotation axis F extends linearly along all the central axes A, B, C, D, and E arranged in a straight line.

Specifically, because the shaft 2, the end effector 3, the joint 7, and the bending operation part 8 are directly or indirectly connected to the rotation operation part 9, they integrally rotate with the rotation operation part 9 about their central axes A, B, C, and D. At this time, because the distal ends and the proximal ends of the four bending wires 10a, 10b, 10c, and 10d are held by the joint 7 and the base part 8a, respectively, the four bending wires 10a, 10b, 10c, and 10d also rotate about the longitudinal axis A at the shaft 2, rotate about the central axis C at the joint 7, rotate about the central axis E at the bending operation part 8, and rotate about the rotation axis F at the rotation operation part 9.

Next, the operation of the treatment instrument 1 will be described.

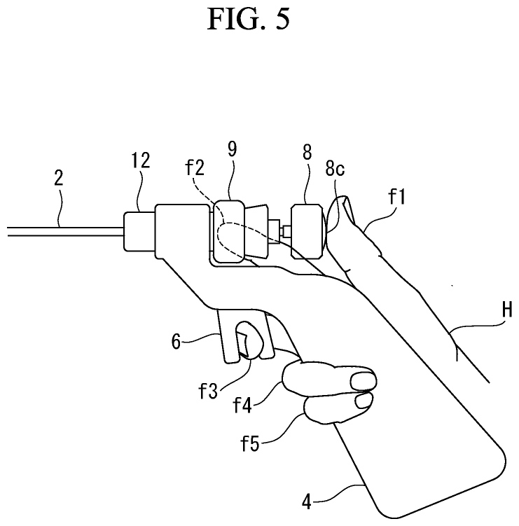

As shown in FIG. 5, an operator grips the grip 4 with one hand H, places the middle finger f3 on the trigger 6, places the index finger f2 on the rotation operation part 9, and places the thumb f1 on the pressing part 8c of the bending operation part 8.

When the operator wishes to bend the joint 7, the operator pushes the bending operation part 8 in a desired direction with the thumb f1. This causes the bending operation part 8 to swivel and tilt about the ball part 8b, which serves as a fulcrum, pulling a part of the bending wires 10a, 10b, 10c, and 10d and generating tension. Then, the joint 7 is bent in response to the tension. For example, as shown in FIGS. 1B and 4A, when the bending operation part 8 is tilted downward, the upper bending wire 10a is tensioned, bending the joint 7 upward. In FIGS. 4A and 4B, tensioned bending wires are hatched.

When the operator wishes to rotate the end effector 3 about its own central axis B, the operator rotates the rotation operation part 9 about the central axis E with the index finger f2. This causes the bending operation part 8, the shaft 2, the joint 7, the end effector 3, and the bending wires 10a, 10b, 10c, and 10d to rotate about the rotation axis F together with the rotation operation part 9. At this time, if a pressing force is applied to the pressing part 8c of the bending operation part 8 by the thumb f1, the base part 8a rotates but the pressing part 8c and the shaft member 8d do not rotate.

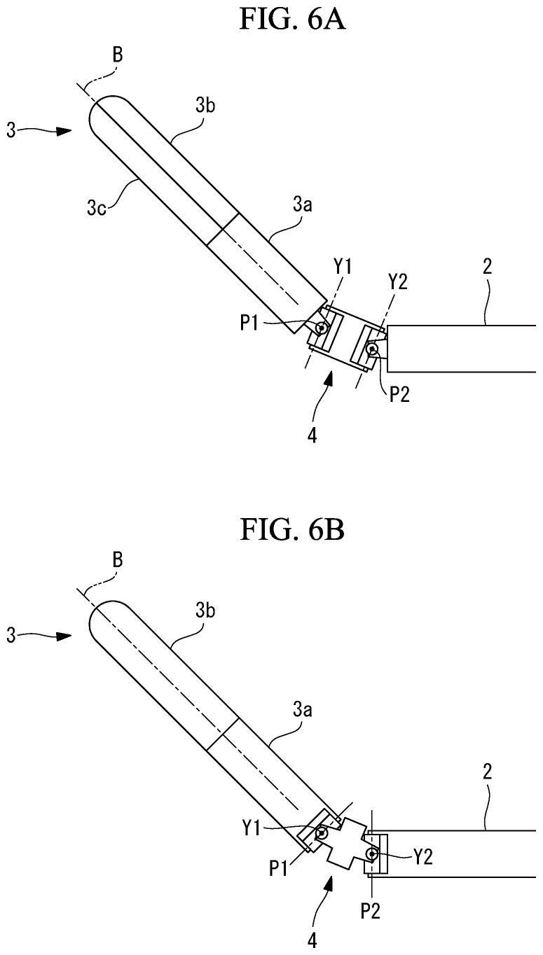

By rotating the four bending wires 10a, 10b, 10c, and 10d in a state in which some of the bending wires 10a, 10b, 10c, and 10d are tensioned, the tensions in the wires are switched among the four bending wires 10a, 10b, 10c, and 10d. Thus, the bending direction and the bending angle of the joint 7 are maintained before and after the rotation. Specifically, as shown in FIGS. 6A and 6B, the end effector 3 rotates about its own central axis B while keeping the bending direction and the bending angle of the joint 7 constant. FIG. 6B illustrates a state in which the end effector 3 has rotated by 90° from the state in FIG. 6A.

FIGS. 4A and 4B show an example of switching of the tensions in the wires. The left drawings in FIGS. 4A and 4B show the positions of the four bending wires 10a, 10b, 10c, and 10d at the joint 7, and the right drawings in FIGS. 4A and 4B show the positions of the four bending wires 10a, 10b, 10c, and 10d at the bending operation part 8. In this example, the four bending wires 10a, 10b, 10c, and 10d are disposed above, below, to the left of, and to the right of the central axes C and D, respectively, and bend the joint 7 up, down, leftward, and rightward, respectively, when tensioned.

As shown in FIG. 4A, by tilting the bending operation part 8 downward with the thumb f1 before rotating the rotation operation part 9, the upper bending wire 10a is tensioned.

FIG. 4B illustrates a state in which the rotation operation part 9 has rotated by 90° to the left, from the state thereof in FIG. 4A. As shown in FIG. 4B, the four bending wires 10a, 10b, 10c, and 10d rotate by 90° to the left at both the bending operation part 8 and the joint 7. As a result, the positions of the four bending wires 10a, 10b, 10c, and 10d are switched. The upper bending wire 10a is moved to the left, and the right bending wire 10d is moved upward. At this time, because the thumb f1 keeps pressing the bending operation part 8 downward, the tension applied to the bending wire 10a before rotation is applied to the bending wire 10d after rotation.

In this way, the tensions in the wires are continuously changed among the four bending wires 10a, 10b, 10c, and 10d while the rotation operation part 9 is rotated. When the rotation operation part 9 has rotated by 180°, the tensions in the bending wires 10a and 10b are completely switched, and the tensions in the bending wires 10c and 10d are completely switched. Specifically, although the positions of the bending wires 10a, 10b, 10c, and 10d change due to the rotation, the tensions in the upper, lower, left, and right bending wires 10a, 10b, 10c, and 10d viewed from the joint 7 and the bending operation part 8 do not change. Hence, as shown in FIGS. 6A and 6B, the bending direction and the bending angle of the joint 7 during and after the rotation of the rotation operation part 9 are maintained to be the same as those before the rotation.

As described above, according to this embodiment, when the rotation operation part 9 is operated in a state in which the joint 7 is bent by operating the bending operation part 8, the four bending wires 10a, 10b, 10c, and 10d rotate integrally with the bending operation part 8 and the joint 7, and the tensions in the wires are switched among the four bending wires 10a, 10b, 10c, and 10d. Thus, it is possible to rotate the end effector 3 about its own rotation axis B while maintaining the bending direction and the bending angle of the joint 7 constant.

Furthermore, this distal roll motion of the end effector 3 is realized without using a roll joint. Hence, it is possible to shorten the length and reduce the cost of the treatment instrument 1.

In addition, the rotation of the rotation operation part 9 is efficiently transmitted to the end effector 3 by the rigid shaft 2 and the rigid joint 7. Hence, the end effector 3 can be rotated responsively to the rotation of the rotation operation part 9. For example, the end effector 3 can be rotated by the same angle as the rotation of the rotation operation part 9.

Because the end effector 3, the joint 7, the shaft 2, and the rotation operation part 9 are arranged in series along the rotation axis F, the rotation of the rotation operation part 9 can be transmitted to the end effector 3 through the shaft 2 and the joint 7. Thus, it is possible to realize rotation of the end effector 3 with a simple configuration without using a complicated mechanism, such as a gear.

In addition, according to this embodiment, the pressing part 8c, which comes into contact with the thumb f1 of the operator who performs the bending operation of the joint 7, and the base part 8a, to which the proximal ends of the bending wires 10a, 10b, 10c, and 10d are fixed, are relatively rotatable about the central axis D. Hence, even in a state in which the pressing part 8c is pressed by the thumb f1, the rotation operation part 9 can be easily rotated. In other words, the bending operation of the joint 7 by the bending operation part 8 and the rotation operation of the end effector 3 by the rotation operation part 9 can be performed simultaneously.

If the pressing part 8c is fixed to the base part 8a, the pressing part 8c pressed by the finger f1 resists the rotation of the rotation operation part 9. Thus, it is difficult to simultaneously perform the bending operation of the joint 7 by the bending operation part 8 and the rotation operation of the end effector 3 by the rotation operation part 9.

Furthermore, according to this embodiment, the rotation operation part 9 is connected to the proximal end of the grip 4, and the bending operation part 8 is connected to the proximal end of the rotation operation part 9. Hence, as shown in FIG. 5, in a state in which the grip 4 is gripped by the palm, the ring finger f4, and the little finger f5, the index finger f2 is positioned at the rotation operation part 9, and the thumb f1 is positioned at the bending operation part 8. Because the pressing part 8c is located at the center of the proximal end face of the bending operation part 8, the thumb f1 is positioned at the pressing part 8c. This allows the operator to easily perform both the bending operation of the joint 7 and the rotation operation of the end effector 3 with only one hand.

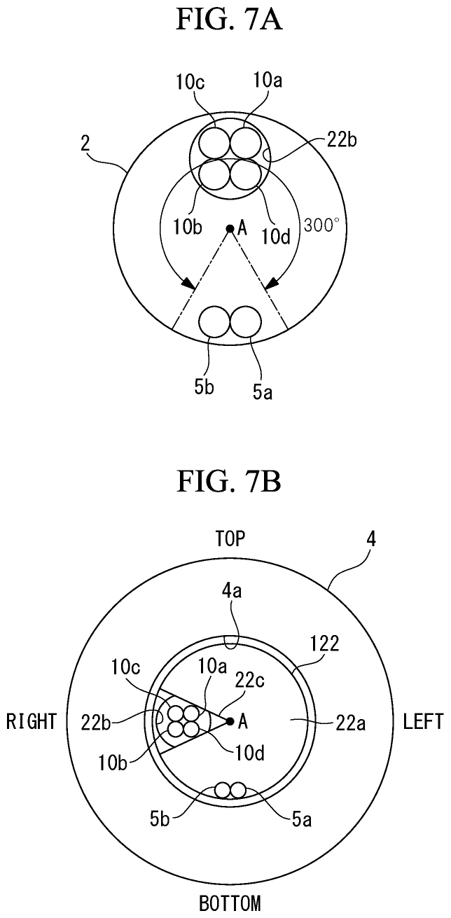

In the above-described embodiment, in order to prevent the four bending wires 10a, 10b, 10c, and 10d from interfering with the opening/closing wires 5a and 5b during rotation of the rotation operation part 9, the four bending wires 10a, 10b, 10c, and 10d are preferably gathered together in one place away from the longitudinal axis A in the radial direction, which intersects the longitudinal axis A, on the proximal side of the shaft 2, as shown in FIG. 7A. In addition, the rotation angle of the rotation operation part 9 may be limited to a predetermined range to limit the rotation angle of the gathered bending wires 10a, 10b, 10c, and 10d to a predetermined range. For example, as shown in FIG. 7A, the rotation angle of the bending wires 10a, 10b, 10c, and 10d may be limited to a range of 300° about the central axis A.

As shown in FIG. 3, the opening/closing wires 5a and 5b passing through the inside of the shaft 2 are led out from the connection member 12 into the grip 4 and extend to the trigger 6.

The connection member 12 is provided with an opening through which the opening/closing wires 5a and 5b are led out. For example, the connection member 12 includes a first member 121 on the distal side, which is fixed to the proximal end of the shaft 2, and a second member 122 on the proximal side, which is fixed to the rotation operation part 9. The shaft 2 and the first member 121 may be formed of a single member, and the rotation operation part 9 and the second member 122 may be formed of a single member. A conical clearance through which the opening/closing wires 5a and 5b pass from the inside to the outside of the connection member 12 is formed between a concave-conical proximal end face 21a of the first member 121 and a convex-conical distal end face 22a of the second member 122.

FIG. 7B is a front view of the second member 122 as viewed from the distal side, in a state in which the first member 121 is removed from the hole 4a. The second member 122 has an accommodation part 22b for gathering the four bending wires 10a, 10b, 10c, and 10d in one place. The accommodation part 22b is a through-hole extending parallel to the longitudinal direction A and formed in a protrusion 22c projecting to the distal side. The four bending wires 10a, 10c, and 10d pass through the accommodation part 22b. The first member 121 and the second member 122 are coupled to each other by a coupling member 13. The first member 121 and the second member 122 may be a single body. The opening/closing wires 5a and 5b are disposed outside the predetermined angular range in which the four bending wires 10b, 10c, and 10d are rotatable.

By doing so, during rotation of the rotation operation part 9, the bending wires 10a, 10b, 10c, and 10d rotating about the rotation axis F are prevented from interfering with the opening/closing wires 5a and 5b. As a result, it is possible to prevent power transmission loss due to interference between the opening/closing wires 5a and 5b and the bending wires 10a, 10b, 10c, and 10d and thus to improve the operability of the end effector 3 and the joint 7. In addition, by gathering the four bending wires 10a, 10b, 10c, and 10d in one place, it is possible to save space and thus to improve the design flexibility.

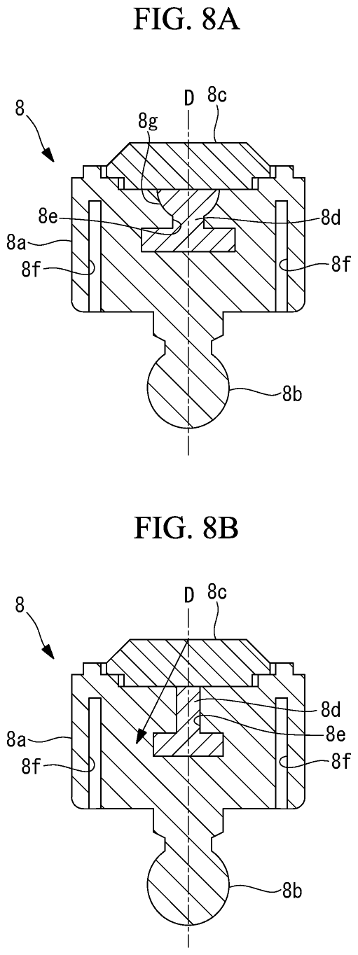

In the above-described embodiment, in the bending operation part 8, a contact surface 8g of the shaft member 8d contacting the base part 8a may be hemispherical, as shown in FIG. 8A.

In the case where the contact surface of the shaft member 8d contacting the base part 8a is a surface parallel to the central axis D, as shown in FIG. 8B, when a pressing force in a direction intersecting the central axis D (see the arrow) is applied to the pressing part 8c, the frictional force between the shaft member 8d and the base part 8a increases. This increases the resistance to the rotation of the base part 8a, reducing the operability of the rotation operation part 9.

The configuration in FIG. 8A can suppress an increase in the frictional force between the shaft member 8d and the base part 8a, as well as an increase in the resistance to the rotation of the base part 8a. Thus, it is possible to realize good operability of the rotation operation part 9 regardless of the direction of the pressing force applied to the pressing part 8c by the operator.

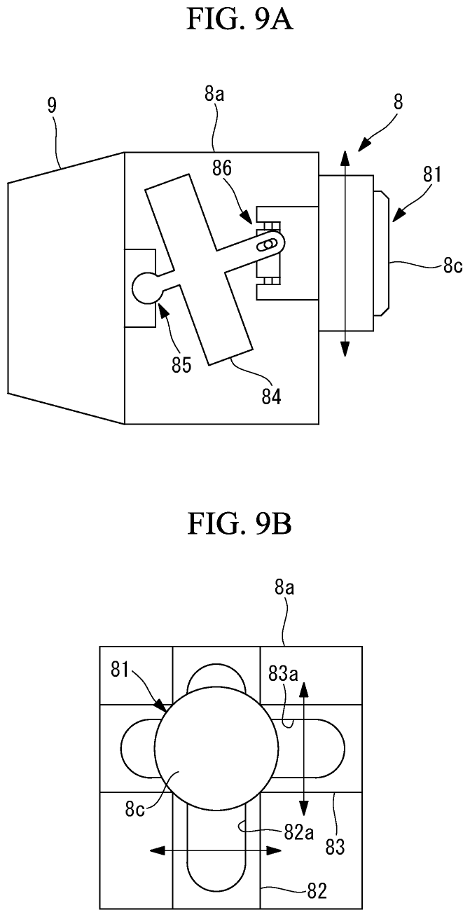

Although the joystick of the bending operation part 8 is swivelable in a direction intersecting the longitudinal direction A, relative to the shaft 2, in the above-described embodiment, instead of this, a joystick 81 including the pressing part 8c may move on a plane perpendicular to the longitudinal direction A of the shaft 2 so as to be parallel to the plane, as shown in FIGS. 9A and 9B.

In the case of the joystick 8 that tilts with respect to the longitudinal axis A, as shown in FIG. 3, the operation of the joystick 8 may be difficult because the thumb tilts together with the joystick 8. For example, when the joystick 8 is tilted downward, the thumb interferes with the grip 4. The joystick 81 that moves parallel to the plane can suppress the inclination of the thumb, thus improving the operability of the joystick 81.

FIGS. 9A and 9B show a configuration example of the bending operation part 8 in which the joystick 81 moves parallel to the plane.

As shown in FIG. 9B, the bending operation part 8 includes a plate-like first guide member 82 having a guide slot 82a for guiding the joystick 81 in the vertical direction, and a plate-like second guide member 83 having a guide slot 83a for guiding the joystick 81 in the horizontal direction. The first guide member 82 is linearly movable in the horizontal direction, and the second guide member 83 is linearly movable in the vertical direction. The joystick 81 is supported by the base part 8a so as to be able to move in a desired direction orthogonal to the central axis D by the guide members 82 and 83. The bending wires 10a, 10b, 10c, and 10d are connected to the joystick 81.

As shown in FIG. 9A, the joystick 81 is coupled to the rotation operation part 9 via a swivel member 84. The swivel member 84 is coupled to the rotation operation part 9 with a spherical bearing 85 and is coupled to the joystick 81 with a gimbal structure 86.

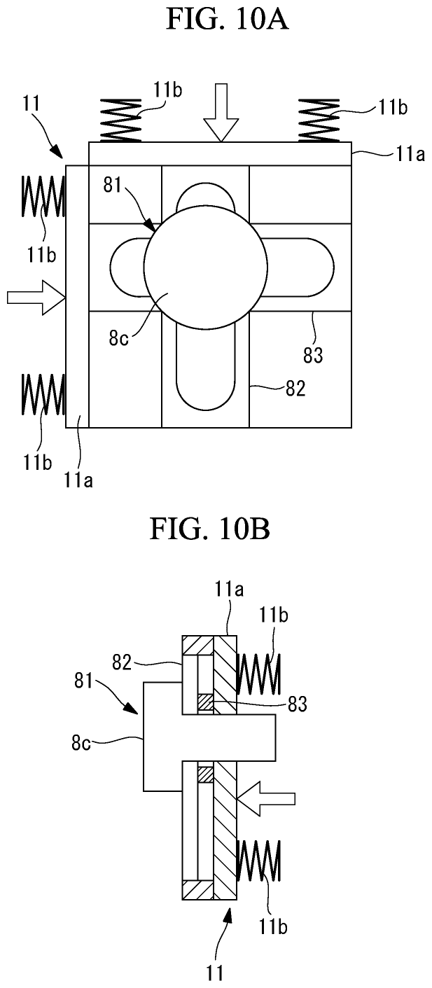

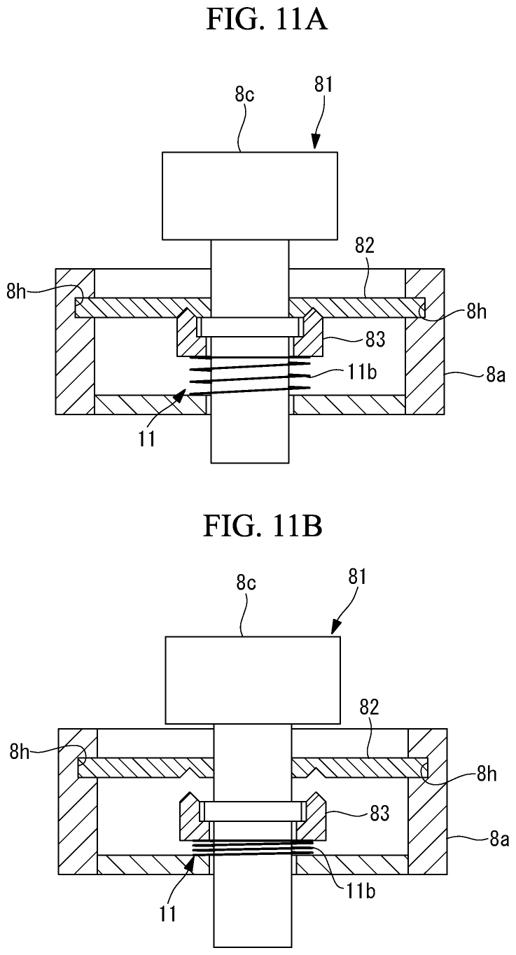

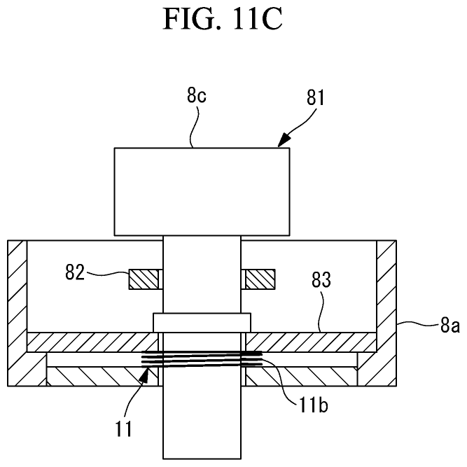

In the above-described embodiment, as shown in FIGS. 10A to 11C, the bending operation part 8 may have a lock mechanism 11 for locking the position of the joystick 81. This configuration allows the joint 7 to remain bent without continuously pressing the joystick 81 with the thumb, thus reducing fatigue of the thumb.

As shown in FIGS. 10A and 10B, an example of the lock mechanism 11 includes fixing members 11a that are in contact with the guide members 82 and 83, and urging members 11b, such as springs, that urge the fixing members 11a toward the guide members 82 and 83.

When no pressing force is applied to the joystick 81, the guide members 82 and 83 are locked by the friction between the guide members 82 and 83 and the fixing members 11a. The operator can displace the joystick 81 while resisting the urging force of the urging members 11b.

In FIG. 10A, the fixing member 11a and the urging members 11b for the first guide member 82 and the fixing member 11a and the urging members 11b for the second guide member 83 are provided. In FIG. 10B, a single fixing member 11a that is in contact with both guide members 82 and 83 is provided.

As shown in FIGS. 11A to 11C, another example of the lock mechanism 11 includes an urging member 11b that urges the guide member 83 toward the guide member 82. The guide member 82 on the pressing part 8c side is supported by a groove 8h formed in the base part 8a so as to be linearly movable in the horizontal direction and so as not to be moved in the direction parallel to the central axis D. In FIGS. 11A to 11C, the vertical direction is the direction parallel to the central axis D. As shown in FIG. 11C, the guide member 83 on the side opposite to the pressing part 8c is movable in the direction parallel to the central axis D. The urging member 11b is disposed on the side of the guide members 82 and 83 opposite to the pressing part 8c.

As shown in FIG. 11A, when no pressing force is applied to the pressing part 8c, the guide members 82 and 83 are locked to each other by the friction between the guide members 82 and 83. As shown in FIGS. 11B and 11C, when a pressing force is applied to the pressing part 8c, the guide member 83 pressed by the pressing part 8c is separated from the guide member 82 while resisting the urging force of the urging member 11b, and the guide members 82 and 83 are unlocked.

In the case of the lock mechanism 11 shown in FIGS. 10A and 10B, the resistance due to the urging force of the urging members 11b is generated in response to the displacement of the joystick 81 in the vertical and horizontal directions. In contrast, in the case of the lock mechanism 11 in FIGS. 11A to 11C, the resistance due to the urging force of the urging member 11b is not generated in response to the displacement of the joystick 81 in the vertical and horizontal directions. Hence, the joystick 81 can be operated with a smaller force, which is advantageous.

Although the bending operation part 8 is disposed on the proximal side of the rotation operation part 9 in the above embodiment, the rotation operation part 9 may be disposed on the proximal side of the bending operation part 8 instead.

In such a case, the configuration of the bending operation part 8 may be appropriately changed so that the operator can easily operate the bending operation part 8 disposed between the proximal end of the shaft 2 and the rotation operation part 9.

Although the end effector 3 is double-action forceps in the above-described embodiment, the type of the end effector 3 is not limited thereto, and the end effector 3 may be any end effector that is generally used in the treatment instrument 1. For example, the end effector 3 may be single-action forceps, a knife, or a camera.

Although the number of bending wires 10a, 10b, 10c, and 10d is four in the above-described embodiment, the number of bending wires is not limited thereto, and may be any number larger than one. In general, the larger the number of the bending wires, the larger the number of degrees of freedom of the bending operation part 8 and the joint 7. Hence, the number of bending wires may be selected depending on the required number of degrees of freedom of the bending operation part 8 and the joint 7.

### REFERENCE SIGNS LIST

1 Treatment instrument
2 Shaft
3 End effector
4 Grip
7 Joint
8 First operation part, Bending operation part
9 Second operation part, Rotation operation part
10a, 10b, 10c, 10d Bending wire
11 Lock mechanism
A, B, C, D, E Central axis
F Rotation axis

1. A treatment instrument comprising:
a shaft;
an end effector disposed on a distal side of the shaft;
a joint connecting the end effector to a distal end of the shaft and being bendable in a direction intersecting a central axis of the shaft;
a first operation part connected to a proximal side of the shaft and configured to bend the joint;
a plurality of wires each connecting the joint and the first operation part through the shaft and each configured to generate tension in response to an operation of the first operation part; and
a second operation part connected to the proximal side of the shaft and configured to integrally rotate the shaft, the end effector, the joint, the first operation part, and the plurality of wires about a rotation axis, which passes through the central axis of the shaft, a central axis of the end effector, a central axis of the joint, and a central axis of the first operation part,
wherein the first operation part includes:
a joystick displaceable in a direction intersecting the central axis of the shaft; and
a lock configured to fix a position of the joystick.

2. The treatment instrument according to claim 1, wherein
the plurality of wires include a first wire and a second wire,
the first wire and the second wire are held on both sides of the central axis of the first operation part at the first operation part and are held on both sides of the central axis of the joint at the joint and,
when the second operation part is operated in a state in which the joint is bent, tensions in the first wire and the second wire are switched.

3. The treatment instrument according to claim 1, wherein
the first operation part includes a pressing part and a rotation part having a face, the pressing part being disposed on the face of the rotation part so as to be supported by the rotation part and being pressed by a finger of an operator, and the pressing part and the rotation part being relatively rotatable about the central axis of the first operation part,
proximal ends of the plurality of wires are fixed to the rotation part, and
the rotation part rotates integrally with the shaft, the end effector, the joint, and the plurality of wires about the rotation axis.

4. The treatment instrument according to claim 2, wherein
the plurality of wires further include a third wire and a fourth wire,
the first wire, the second wire, the third wire, and the fourth wire are held at four equally or substantially equally spaced positions about the central axis of the first operation part at the first operation part and are held at four equally or substantially equally spaced positions about the central axis of the joint at the joint, and
the joint is bendable in any direction intersecting the central axis of the shaft.

5. The treatment instrument according to claim 1, wherein the joystick is provided on a proximal side of the second operation part.

6. The treatment instrument according to claim 4, wherein the first wire, the second wire, the third wire, and the fourth wire are gathered together in one place away from the central axis of the shaft in a direction intersecting the central axis, on the proximal side of the shaft.

7. The treatment instrument according to claim 1, wherein the joystick is movable on a plane extending in a direction perpendicular to the central axis of the shaft so as to be parallel to the plane.

8. A treatment instrument comprising:
a shaft;
an end effector disposed on a distal side of the shaft;
a joint connecting the end effector to a distal end of the shaft and being bendable in a direction intersecting a central axis of the shaft;
a first operation part connected to a proximal side of the shaft and configured to bend the joint;
a plurality of wires each connecting the joint and the first operation part through the shaft and each configured to generate tension in response to an operation of the first operation part; and
a second operation part connected to the proximal side of the shaft and configured to integrally rotate the shaft, the end effector, the joint, the first operation part, and the plurality of wires about a rotation axis, which passes through the central axis of the shaft, a central axis of the end effector, a central axis of the joint, and a central axis of the first operation part,
wherein the first operation part includes a joystick displaceable in a direction intersecting the central axis of the shaft and movable on a plane extending in a direction perpendicular to the central axis of the shaft so as to be parallel to the plane.

9. The treatment instrument according to claim 8, wherein
the plurality of wires include a first wire and a second wire,
the first wire and the second wire are held on both sides of the central axis of the first operation part at the first operation part and are held on both sides of the central axis of the joint at the joint and,
when the second operation part is operated in a state in which the joint is bent, tensions in the first wire and the second wire are switched.

10. The treatment instrument according to claim 8, wherein
the first operation part includes a pressing part and a rotation part having a face, the pressing part being disposed on the face of the rotation part so as to be supported by the rotation part and being pressed by a finger of an operator, and the pressing part and the rotation part being relatively rotatable about the central axis of the first operation part,
proximal ends of the plurality of wires are fixed to the rotation part, and
the rotation part rotates integrally with the shaft, the end effector, the joint, and the plurality of wires about the rotation axis.

11. The treatment instrument according to claim 9, wherein
the plurality of wires further include a third wire and a fourth wire,
the first wire, the second wire, the third wire, and the fourth wire are held at four equally or substantially equally spaced positions about the central axis of the first operation part at the first operation part and are held at four equally or substantially equally spaced positions about the central axis of the joint at the joint, and
the joint is bendable in any direction intersecting the central axis of the shaft.

12. The treatment instrument according to claim 8, wherein the joystick is provided on a proximal side of the second operation part.

13. The treatment instrument according to claim 11, wherein the first wire, the second wire, the third wire, and the fourth wire are gathered together in one place away from the central axis of the shaft in a direction intersecting the central axis, on the proximal side of the shaft.

14. The treatment instrument according to claim 8, wherein the first operation part further includes a lock configured to fix a position of the joystick.

15. A treatment instrument comprising:
a shaft;
an end effector disposed on a distal side of the shaft;
a joint connecting the end effector to a distal end of the shaft and being bendable in a direction intersecting a central axis of the shaft;
a first operation part connected to a proximal side of the shaft and configured to bend the joint;
a first wire, a second wire, a third wire, and a fourth wire each connecting the joint and the first operation part through the shaft and each configured to generate tension in response to an operation of the first operation part; and
a second operation part connected to the proximal side of the shaft and configured to rotate the end effector about a central axis of the end effector, wherein
the first wire, the second wire, the third wire, and the fourth wire are held at four equally or substantially equally spaced positions about a central axis of the first operation part at the first operation part and are held at four equally or substantially equally spaced positions about a central axis of the joint at the joint, and
the first wire, the second wire, the third wire, and the fourth wire are gathered together in one place away from the central axis of the shaft in a direction intersecting the central axis, on the proximal side of the shaft.

16. The treatment instrument according to claim 15, wherein
when the second operation part is operated in a state in which the joint is bent, tensions in the first wire and the second wire are switched.

17. The treatment instrument according to claim 15, wherein
the first operation part includes a pressing part and a rotation part having a face, the pressing part being disposed on the face of the rotation part so as to be supported by the rotation part and being pressed by a finger of an operator, and the pressing part and the rotation part being relatively rotatable about the central axis of the first operation part,
proximal ends of the plurality of wires are fixed to the rotation part, and
the rotation part rotates integrally with the shaft, the end effector, the joint, and the plurality of wires about the rotation axis.

18. The treatment instrument according to claim 17, wherein the first operation part includes a joystick displaceable in a direction intersecting the central axis of the shaft and is provided on a proximal side of the second operation part.

19. The treatment instrument according to claim 18, wherein the joystick is movable on a plane extending in a direction perpendicular to the central axis of the shaft so as to be parallel to the plane.

20. The treatment instrument according to claim 18, wherein the first operation part further includes a lock configured to fix a position of the joystick.
